(19) United States
(12) Patent Application Publication
(10) Pub. No.: US 2025/0312518 A1
(43) Pub. Date: Oct. 9, 2025

(54) HYDROGELS

(71) Applicant: The University of Liverpool, Liverpool (GB)

(72) Inventors: Rachel Williams, Liverpool (GB); He Liang, Liverpool (GB); Hannah Levis, Liverpool (GB); Vito Romano, Liverpool (GB)

(21) Appl. No.: 18/853,549

(22) PCT Filed: Apr. 6, 2023
(86) PCT No.: PCT/EP2023/059282

§ 371 Date: Oct. 2, 2024

(30) Foreign Application Priority Data
Apr. 6, 2022 (GB) 2205071.0

(51) Int. Cl. A61L 27/52; A61L 27/18

(52) U.S. Cl. CPC A61L 27/52; A61L 27/18; A61L 2430/16

(57) ABSTRACT

The present invention relates to hydrogels, methods of making them, their use in the treatment of eye disorders and as bandage contact lenses. The present invention also provides modified poly-ε-lysine polymers and methods of making them.

### RELATED APPLICATIONS

This application is the § 371 National Stage of PCT/EP23/59282, filed Apr. 6, 2023, which claims the benefit of priority to GB Application 2205071.0, filed Apr. 6, 2022. The contents of PCT/EP23/59282 are fully incorporated herein by reference.

### FIELD OF THE INVENTION

The present invention relates to hydrogels, methods of making them, their use in the treatment of eye disorders and as bandage contact lenses. The present invention also provides modified poly-ε-lysine polymers and methods of making them.

### BACKGROUND OF THE INVENTION

Corneal diseases are the fifth leading cause of blindness in the world and a significant subset of corneal disease is due to dysfunction of the corneal endothelium [Pascolini D, Mariotti S P Global estimates of visual impairment: 2010 British Journal of Ophthalmology 2012; 96:614-618]. The corneal endothelium is responsible for preserving the overall transparency of the cornea by maintaining a homeostatic balance of hydration levels. Endothelial dysfunction is the most common indication for corneal transplantation, however, the number of available corneal tissues is limited worldwide. Globally, there are an estimated 12.7 million people waiting for a corneal transplant, which equates to only one cornea available for every 70 required [Gain P et al. Global Survey of Corneal Transplantation and Eye Banking. JAMA Ophthalmol. 2016; 134 (2): 167-173]. Even when donation is high in a particular region, approximately one third of harvested donor tissues are not suitable for transplant due to low endothelial cell count or presence of infectious agents upon screening. The success of cadaveric donor transplantation is limited by the long-term risk of graft failure. 30% of corneal endothelial cells are lost from the graft within the first 6 months of transplantation, which can lead to graft failure. The demand for corneal tissues will only increase proportionally together with an ageing population and waiting times of up to 2 years already severely affect patients' quality of life.

Diseases of the corneal endothelium, such as Fuchs' endothelial corneal dystrophy (FECD) and pseudophakic bullous keratopathy (PBK), result in significant loss of vision and are the commonest reasons for corneal transplantation. Damage to this endothelial layer leads to oedema and thus loss of transparency. Replacement of the endothelial layer with a corneal transplant reduces the oedema and restores transparency. Although corneal transplantation is more than 80% successful at one year, five-year graft survival rates are significantly reduced to 70% for FECD and 52% for PBK, meaning that patients often require a second transplant. There is a global shortage of corneas with only one available for every 70 required. Therefore, there is an opportunity to combine biomaterials with in vitro expanded corneal endothelial cells (CECs) to produce multiple bioengineered grafts from each donor cornea. These biomaterials not only serve as a carrier for CEC transplantation but may also enhance cell function to increase the long-term success of transplanted grafts.

In recent years, lamellar techniques have overtaken full thickness penetrating keratoplasty (PK) transplants as the most common surgical procedure to treat corneal endothelial failure. Endothelial keratoplasty (EK) includes Descemet's stripping automated endothelial keratoplasty (DSAEK), where the endothelial layer and underlying Descemet's membrane attached to a portion of posterior stroma (approx. 100 μm) are transplanted. The addition of the stromal portion results in easier handling as the stiffness of the graft is increased. Another EK technique is Descemet's membrane endothelial keratoplasty (DMEK), which transplants only the endothelial layer and the underlying 10-15 μm Descemet's membrane (DM). The advantage of EK is that suture related infection and inflammation and secondary immune reaction are minimised. Additionally, graft rejection is reduced in DSAEK compared to PK and significantly reduced in DMEK compared to PK [Hos D et al. Immune reactions after modern lamellar (DALK, DSAEK, DMEK) versus conventional penetrating corneal transplantation. Prog Retin Eye Res. 2019 73:100768]. In low risk PK the risk of endothelial immune rejection is 5-17% within the first 2 years whereas for DSAEK it is 8-14%. This reduced risk is thought to be due to several factors. The graft is introduced into the anterior chamber, therefore, the mechanisms of anterior chamber associated immune deviation (ACAID) may contribute. ACAID is a well-known phenomenon where alloantigens introduced into the anterior chamber lead to a systemic and antigen-specific suppression of the immune responses [Niederkorn J Y. The immune privilege of corneal allografts. Transplantation. 1999 27; 67 (12): 1503-8]. The exposure of the recipient cornea to antigen presenting cells (APCs), which are predominantly located in the anterior stroma, is reduced. Equally, fewer of the APCs are transplanted and because the epithelium and majority of the stroma is not transplanted, the whole tissue is likely to be less immunogenic [Hos D et al, 2019]. The risk of graft rejection after DMEK is minimal at 0.9% at 1 year and 2.3% at 4 years [Price M O et al. Descemet's membrane endothelial keratoplasty surgery: update on the evidence and hurdles to acceptance. Curr Opin Ophthalmol. 2013; 24 (4): 329-35]. Note that only DM and the endothelial layer are transplanted in these grafts. DMEK has also been shown to produce faster recovery times and better visual outcomes, [Hos D et al. Incidence and Clinical Course of Immune Reactions after Descemet Membrane Endothelial Keratoplasty: Retrospective Analysis of 1000 Consecutive Eyes. Ophthalmology. 2017; 124 (4): 512-518] however, this procedure is only being slowly adopted, likely due to the increased surgical skill required to prepare and transplant such a graft. Engineering a bio-synthetic graft that combines the visual outcomes and immune response of DMEK grafts with the handle-ability of DSAEK grafts could produce a significant medical advancement in this field, as well as reducing the burden of the global tissue shortage.

Hydrogels can be used for tissue engineering of the layers of the cornea (epithelium, stroma and endothelium).

### Endothelium

Endothelial dysfunction is the most common indication for corneal transplantation. Due to a global corneal donor shortage, meaning only one donor corneal is available for every 70 required, there are over 12 million people on the waiting list for a corneal transplant (Gain P, Jullienne R, He Z, Aldossary M, Acquart S, Cognasse F, Thuret G. Global Survey of Corneal Transplantation and Eye Banking. JAMA Ophthalmol. 2016 February; 134 (2): 167-73). Tissue engineering corneal endothelial tissues by culturing expanded corneal endothelial cells (CECs) on a carrier material is a solution to this problem.

Others have developed tissue engineered corneal endothelial grafts using cultured CECs combined with synthetic scaffolds. The majority of these competing solution have only reached the in vitro stage largely because although the materials may show good cell compatibility, they lack the mechanical properties required to form a clinically useful graft that can withstand manipulation, or vice versa.

The ideal material to form the basis of a tissue engineered corneal endothelial graft would allow transmission of light (wavelength 400-780 nm), be of a thickness between 10 μm and 100 um (preferably 10-50 um) and have sufficient mechanical properties (tensile strength, Young's modulus) that allow for handling with forceps and manipulation into clinical graft delivery devices (scrolled to be inserted through a 4 mm incision). The material is not required to degrade as the graft can stay in place indefinitely (as is the case with corneal transplant tissue grafts). It would need to allow attachment of CECs, formation of a monolayer of cells and regulation of cell behaviour i.e. fluid transport in and out of the stroma, so the material must also be permeable.

### Epithelium

The corneal epithelium is the outermost layer of the cornea and is constantly renewed by a population of stem cells in the limbal region at the periphery of the cornea. These cells can be diseased or damaged by physical injury leading to dysfunctional renewal of the ocular surface. Treatment to repair the ocular surface can be by transplant of the limbal stem cells on a carrier material or transfer of epithelial cells to the corneal surface (material does not need to remain on the surface once the cells have transferred).

This would require a material that allowed attachment and growth of a monolayer of epithelial cells (and subsequent detachment if transferring cells to the surface of the cornea.) The material should have the mechanical properties sufficient for manipulation with forceps and flexibility to conform to the surface curvature of the cornea (or ability to be moulded into a dome shape). If used for transfer the material would not need to be transparent and can be designed to act as a bandage contact lens to protect the transferring cells in the initial stages. If remaining in place it should support the remodelling by epithelial cells and be able to withstand the mechanical forces of eyelid motion.

### Stroma

Disease, damage or scarring in the corneal stroma often necessitates a corneal transplant, which can either be full thickness (penetrating keratoplasty) or partial thickness (anterior lamellar keratoplasty, or deep anterior lamellar keratoplasty). All of the transplant solutions require cadaveric donor tissue so tissue engineering solutions are also being investigated for the corneal stroma.

The ideal material to form the basis of tissue engineered stromal tissue would be able to transmit light (wavelength 400-780 nm), be mechanically strong to maintain its structural integrity under intraocular pressure, as well as forces exerted on it such as eyelid and tear film motion. It must have good mechanical integrity for handling with forceps and suturing. But it is worth noting that Young's modulus and tensile strength of cornea vary considerably between publications (modulus≈100 kPa to 57 MPa; strength≈3-6 MPa) (Ahearne, M., Fernández-Perez, J., Masterton, S., Madden, P. W., Bhattacharjee, P., Designing Scaffolds for Corneal Regeneration. Adv. Funct. Mater. 2020, 30). The material does not need to degrade but it should support the cells to remodel it into a tissue that more accurately resembles the cornea. Extracellular matrix molecules could enhance/replace the structure over time without compromising integrity. It should not adversely refract light and would need to be permeable and porous to allow fluid regulation and cell migration through the structure.

For the production of a full thickness (approximately 500 um) stroma processes like bioprinting (extrusion, inkjet of laser based and stereolithographic) can be employed; so the material should ideally be compatible with these processes.

There is, therefore, a need for improved approaches for ophthalmic tissue engineering and methods to produce hydrogels for that purpose.

The present invention was devised with the foregoing in mind.

### SUMMARY OF THE INVENTION

The present invention provides modified poly-ε-lysine (peK) hydrogels and methods of making said hydrogels. The present invention also provides modified poly-ε-lysine polymers and methods of making said polymers.

In one aspect, there is provided a method for making a hydrogel, the method comprising:
i) providing a solution comprising a modified poly-ε-lysine polymer which comprises a repeating monomer unit according to formula (A) below:

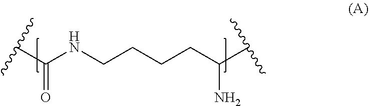

wherein from 4.5% to 75% of the amino groups have been functionalised with pendant functional acrylate groups; and
ii) exposing the modified poly-ε-lysine polymer to UV light to crosslink the modified poly-ε-lysine and provide the hydrogel.

According to another aspect of the invention, there is provided a hydrogel comprising a polymer which comprises a repeating monomer unit according to formula (A) below:

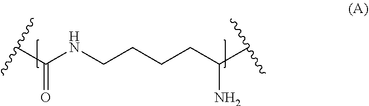

wherein from 10% to 75% of the amine groups are functionalised with an acrylate group of the formula:

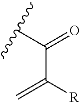

or are crosslinked by a group of the formula:

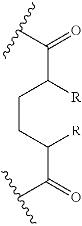

wherein each R is selected from hydrogen or (1-4C)alkyl.

In another aspect, the present invention provides a bandage contact lens comprising a hydrogel as defined herein. Suitably, the hydrogel forming the bandage contact lens comprises cross-linked blend of modified poly-ε-lysine and poly(ethylene glycol) diacrylate polymers as described herein. In another aspect, the present invention provides a hydrogel or bandage contact lens as defined herein, for use in therapy.

In another aspect, the present invention provides a hydrogel or bandage contact lens as defined herein, for use in the treatment of disease or damage to the corneal epithelium or corneal endothelium.

In another aspect, the present invention provides a hydrogel or bandage contact lens as defined herein, for use in the treatment of persistent corneal defects, limbal stem cell deficiency, microbial keratitis, post corneal surgery or to aid healing following post corneal cross-linking for keratoconus.

In another aspect, the present invention provides a hydrogel or bandage contact lens as defined herein, for use in the treatment of corneal swelling due corneal oedema (e.g. bullous keratopathy, Fuchs Endothelial Dystrophy, Congenital Hereditary Endothelial Dystrophy, hydrops of the cornea in keratoconus.

In another aspect, the present invention provides a hydrogel or bandage contact lens as defined herein, for use:
a. as an insert to support a damaged cornea;
b. as a transplant to replace part of a damaged cornea;
c. as a stromal replacement in the treatment of keratoconus;
d. in re-shaping of the cornea, for example reshaping a donor cornea for transplantation;
e. in stabilising corneal shape.

In another aspect, the present invention provides the use of a hydrogel as defined herein, as a support for cell growth (e.g. as a scaffold for corneal endothelial cells).

In another aspect, the present invention provides a method of treating a collagenic eye disorder, said method comprising applying a hydrogel or bandage contact lens as defined herein to the eye of a subject in need of such treatment.

In another aspect, the present invention provides a method of treating disease or damage to the corneal epithelium or corneal endothelium, said method comprising applying a hydrogel or bandage contact lens as defined herein defined herein to the eye of a subject in need of such treatment.

In another aspect, there is provided a modified poly-ε-lysine polymer which comprises a repeating monomer unit according to formula (A) below:

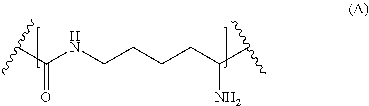

wherein from 4.5% to 75% of the amino groups have been functionalised with pendant functional acrylate groups.

In another aspect, there is provided a modified poly-ε-lysine polymer which comprises a repeating monomer unit according to formula (A) below:

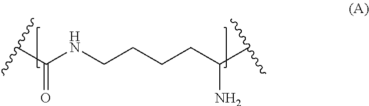

wherein from 10% to 75% of the amino groups have been functionalised with pendant functional acrylate groups.

In another aspect, there is provided a method of preparing a modified poly-ε-lysine, the method comprising reacting poly-ε-lysine with an acrylic acid derivative or an acrylic acid anhydride derivative.

### DETAILED DESCRIPTION OF THE INVENTION

### Definitions

Poly-ε-lysine is a poly(amino acid) which comprises a repeating unit with the structure:

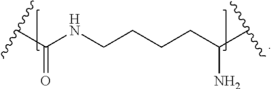

In the present invention, there is provided a modified poly-ε-lysine in which a proportion of the amino groups are functionalised as defined herein.

In the context of the present invention, references to the percentage of amino groups which are functionalised refers to the stoichiometric percentage.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

Unless otherwise stated, the following terms used in the specification and claims have the following meanings set out below.

The terms “treating” or “treatment” include prophylaxis as well as the alleviation of established symptoms of a disease or condition, including pain or discomfort. “Treating” or “treatment” therefore includes: (1) preventing or delaying the appearance of clinical symptoms of the disease or condition developing in a subject that may be afflicted with or predisposed to the disease or condition, but does not yet experience or display clinical or subclinical symptoms of the disease or condition, (2) inhibiting the disease or condition, i.e., arresting, reducing or delaying the development of the disease or condition or a relapse thereof (in case of maintenance treatment) or at least one clinical or subclinical symptom thereof, or (3) relieving or attenuating the disease or condition, i.e., causing regression of the disease or condition or at least one of its clinical or subclinical symptoms.

Unless otherwise specified, where the quantity or concentration of a particular component of a given formulation is specified as a weight percentage (wt. % or % w/w), said weight percentage refers to the percentage of said component by weight relative to the total weight of the formulation as a whole. It will be understood by those skilled in the art that the sum of weight percentages of all components of a formulation will total 100 wt. %. However, where not all components are listed (e.g. where formulations are said to “comprise” one or more particular components), the weight percentage balance may optionally be made up to 100 wt % by unspecified ingredients (e.g. a diluent, such as water, or other non-essential but suitable additives).

In this specification the term “alkyl” includes both straight and branched chain alkyl groups. References to individual alkyl groups such as “propyl” are specific for the straight chain version only and references to individual branched chain alkyl groups such as “isopropyl” are specific for the branched chain version only. For example, “(1-6C)alkyl” includes (1-4C)alkyl, (1-3C)alkyl, propyl, isopropyl and t-butyl.

The term “(m-nC)” or “(m-nC) group” used alone or as a prefix, refers to any group having m to n carbon atoms.

Unless otherwise stated, the term “collagenic eye disorder” refers to eye disorders that are associated with the weakening, degradation and/or damage to structural proteins, such as collagen, in the eye. Although it will be appreciated by a person skilled in the art that collagen is the main structural protein referred to herein, it will be understood that the term “collagenic eye disorder” also encompasses eye disorders associated with the weakening, degradation and/or damage of collagen in combination with other structural proteins in the eye. Furthermore, the term encompasses the weakening, degradation and/or damage to all parts of the eye, such as, for example, the cornea and the sclera.

### Methods of Making Hydrogels of the Present Invention

As described herein, the present invention provides a method for making a hydrogel, the method comprising:
i) providing a solution comprising a modified poly-ε-lysine polymer which comprises a repeating monomer unit according to formula (A) below:

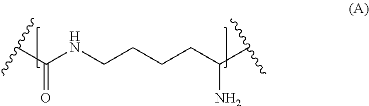

wherein from 10% to 75% of the amino groups have been functionalised with pendant functional acrylate groups; and
ii) exposing the modified poly-ε-lysine to UV light to crosslink the modified poly-ε-lysine and provide the hydrogel.

Suitably, from 20% to 60% of the amino groups in the modified poly-ε-lysine are functionalised with pendant functional acrylate groups, more suitably from 20% to 50% of the amino groups are functionalised with pendant functional acrylate groups.

Suitably, from 10% to 75% of the amino groups have been functionalised with pendant functional acrylate groups. More suitably, from 15% to 40% of the amino groups have been functionalised with pendant functional acrylate groups. More suitably, from 20% to 50% of the amino groups have been functionalised with pendant functional acrylate groups. Most suitably, from 20% to 60% of the amino groups have been functionalised with pendant functional acrylate groups.

Suitably, from 12% to 75% of the amino groups have been functionalised with pendant functional acrylate groups. More suitably, from 12% to 60% of the amino groups have been functionalised with pendant functional acrylate groups. Most suitably, from 12% to 40% of the amino groups have been functionalised with pendant functional acrylate groups.

Suitably, from 30% to 75% of the amino groups have been functionalised with pendant functional acrylate groups. More suitably, from 30% to 60% of the amino groups have been functionalised with pendant functional acrylate groups. Most suitably, from 30% to 50% of the amino groups have been functionalised with pendant functional acrylate groups.

The modified poly-ε-lysine polymer may be any modified poly-ε-lysine polymer as defined herein.

Suitably the solution comprising the modified poly-ε-lysine is an aqueous solution.

The solution may be formed by adding the modified poly-ε-lysine to deionised water.

The solution may further comprise a buffer to maintain the pH. Suitably, the buffer is phosphate buffered saline.

The solution may further comprise a photoinitiator. The photoinitiator may be selected from 2-hydroxy-40-(2-hydroxyethoxy)-2-methylpropiophenone (12959), lithium phenyl-2,4,6-trimethylbenzoylphosphinate (LAP), ethyl (2,4,6-trimethylbenzoyl)phenyl phosphinate (TPO-L) or bis(2,4,6-trimethylbenzoyl)-phenylphosphineoxide (Irgacure 819).

Suitably, the photo initiator is present in the solution in an amount of from 0.05 to 5 wt. %, more suitably from 0.1 to 2 wt. %, most suitably from 0.5 to 1.5 wt. %.

Suitably, the method further comprises casting the solution of modified poly-ε-lysine into a film prior to exposure to UV light.

Suitably, the UV light has a wavelength of from 250 to 405 nm, for example from 260 to 365 nm, or from 345 to 405 nm.

Suitably, the modified poly-ε-lysine is formed by reacting poly-ε-lysine with an acrylic acid derivative or an acrylic acid anhydride derivative. Suitably, the acrylic acid derivative or an acrylic acid anhydride derivative is selected from acrylic acid, methacrylic acid, acrylic anhydride or methacrylic anhydride.

Suitably, the poly-ε-lysine used to form the modified poly-ε-lysine has a molecular weight of from 2000 to 8000 Da, more suitably from 3000 to 7000 Da, most suitably from 4000 to 6000 Da.

Suitably, the poly-ε-lysine has a degree of polymerisation of from 10 to 35, for example 15 to 30 or 15 to 25.

Suitably, the poly-ε-lysine used to form the modified poly-ε-lysine has a degree of polymerisation of from 25 to 35.

Suitably, the solution may comprise the modified poly-ε-lysine in an amount of from 5 to 35 wt. %, more suitably from 10 to 25 wt. %.

Suitably, the solution has a pH in the range of 6 to 8.5, more suitably the solution has a pH in the range of 7 to 8.

The modified poly-ε-lysine may be exposed to UV light in the presence of a poly(ethylene glycol) diacrylate to form a hydrogel comprising a crosslinked polymer blend.

The solution may further comprise a poly(ethylene glycol) diacrylate polymer. The poly(ethylene glycol) diacrylate polymer may be poly(ethylene glycol) dimethacrylate or poly(ethylene glycol) diacrylate.

Suitably, the poly(ethylene glycol) diacrylate polymer is present in the solution in a concentration of from 0.1 to 20 wt. %. More suitably, the poly(ethylene glycol) diacrylate polymer is present in a concentration of from 0.5 to 10 wt. %. Most suitably, the poly(ethylene glycol) diacrylate polymer is present in a concentration of from 1 to 7.5 wt. %.

The weight ratio of modified poly-ε-lysine to poly(ethylene glycol) polymer in the solution may be from 0.5:1 to 50:1, from 1:1 to 40:1, from 2:1 to 30:1 or more suitably from 5:1 to 25:1. In a particular embodiment, the weight ratio of modified poly-ε-lysine to poly(ethylene glycol) diacrylate polymer in the solution be from 1:1 to 5:1, for example 1:1, 2:1, 3:1, 4:1 or 5:1.

Suitably, the poly(ethylene glycol) diacrylate polymer has a molecular weight of from 500 to 15000 Da, more suitably from 1000 to 12000 Da, most suitably from 6000 to 10000 kDa (e.g. 8000 Da).

Suitably, a solution comprising both the modified poly-ε-lysine polymer and the poly(ethylene glycol) diacrylate polymer is exposed to UV light to form a crosslinked polymer blend.

### Hydrogels of the Present Invention

As previously stated, the present invention provides a hydrogel obtained by, obtainable by, or directly obtained by the method of the present invention.

Thus, the present provides a hydrogel formed by UV crosslinking of a modified poly-ε-lysine (PEK) polymer as defined herein. The PEK hydrogel therefore comprises a crosslinked modified poly-ε-lysine polymer.

The UV crosslinking of a modified poly-ε-lysine polymer as defined herein will result in the formation of crosslinks between repeating monomer units, wherein the crosslinks have the formula:

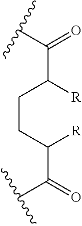

wherein each R is selected from hydrogen or (1-4C)alkyl.

The present invention therefore also provides a hydrogel comprising a polymer which comprises a repeating monomer unit according to formula (A) below:

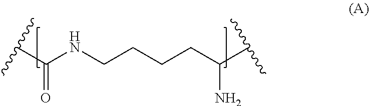

wherein 10% to 75% of the amine groups are functionalised with an acrylate group of the formula:

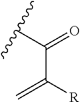

or are crosslinked by a group of the formula:

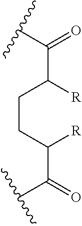

wherein each R is selected from hydrogen or (1-4C)alkyl.

Suitably, each R group in either the acrylate group or crosslinking group is hydrogen or methyl.

Suitably, the hydrogel is in the form of a film.

Suitably, a hydrogel film as defined herein may have a thickness of from 10 to 500 microns.

Suitably, if the hydrogel is to be used in tissue engineering applications, it may have a film thickness of from 10 to 100 microns, more suitably from 10 to 50 microns.

Suitably, a bandage contact lens comprising a hydrogel as defined herein will have a thickness of from 50 to 400 microns, or more suitably, from 70 to 300 microns.

Suitably, the hydrogel has a water content of at least 70 wt. %, or more suitably 80 wt. %

Certain applications of the hydrogels of the present invention require a high degree of transparency. Therefore, in some embodiments the hydrogels of the present invention have a light transmittance of at least 80%. However, not all applications of the hydrogels of the present invention will require a high degree of transparency. In some applications of the invention the transparency is not relevant to the suitability of the hydrogel.

In certain embodiments, the hydrogel comprises only modified poly-ε-lysine as the gel forming component. However, in other embodiments, the hydrogel may further comprise one or more additional polymers.

### Polymer Blends

As described herein, the modified poly-ε-lysine may be exposed to UV light in the presence of a poly(ethylene glycol) diacrylate to form a hydrogel comprising a crosslinked polymer blend.

Suitably, the hydrogel further comprises a poly(ethylene glycol) diacrylate. The poly(ethylene glycol) diacrylate may be selected from poly(ethylene glycol) diacrylate or poly(ethylene glycol) dimethacrylate.

Poly(ethylene glycol) diacrylate or poly(ethylene glycol) dimethacrylate have the following formula:

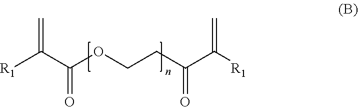

wherein each R₁ is either hydrogen or methyl. Suitably, R₁ is hydrogen.

Suitably, the poly(ethylene glycol) diacrylate may be crosslinked to the modified poly-ε-lysine of the hydrogel. Thus, the hydrogel may comprise a cross-linked blend of modified poly-ε-lysine and poly(ethylene glycol) diacrylate polymers. Thus, the hydrogel suitably further comprises a poly(ethylene glycol) which comprises a repeating unit according to formula (B′) below:

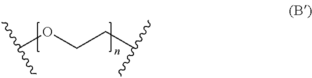

wherein the poly(ethylene glycol) is crosslinked at both of the terminal positions to the modified poly-ε-lysine polymer.

Suitably, the crosslinking group between the repeating unit of Formula B′ and the modified poly-ε-lysine polymer has the formula:

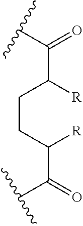

wherein each R is selected from hydrogen or (1-4C)alkyl. Suitably, R is hydrogen or methyl.

The weight ratio of modified poly-ε-lysine to poly(ethylene glycol) polymer in the hydrogel may be from 0.5:1 to 50:1, from 1:1 to 40:1, from 2:1 to 30:1 or more suitably from 5:1 to 25:1.

In a particular embodiment, the weight ratio of modified poly-ε-lysine to poly(ethylene glycol) diacrylate polymer in the hydrogel is from 1:1 to 5:1, for example 1:1, 2:1, 3:1, 4:1 or 5:1.

Suitably, the weight ratio of modified poly-ε-lysine to poly(ethylene glycol) diacrylate polymer in the hydrogel is from 20:80 to 80:20, for example, 20:80, 30:70, 40:60, 50:50, 60:40, 70:30 or 80:20 or ratios in-between.

Suitably, the weight ratio of modified poly-ε-lysine to poly(ethylene glycol) diacrylate polymer in the hydrogel is from 40:60 to 70:30, for example 40:60, 50:50, 60:40 or 70:30 or ratios in-between. More suitably, the weight ratio of modified poly-ε-lysine to poly(ethylene glycol) diacrylate polymer in the hydrogel is from 40:60 to 60:40.

Suitably, the weight ratio of modified poly-ε-lysine to poly(ethylene glycol) diacrylate polymer in the hydrogel is from 50:50 to 70:30, for example, 50:50, 60:40 or 70:30, or ratios in-between.

In an embodiment, the weight ratio of modified poly-ε-lysine to poly(ethylene glycol) diacrylate polymer in the hydrogel is from 40:60 to 80:20; and from 4.5% to 75% of the amino groups have been functionalised with pendant functional acrylate groups.

In an embodiment, the weight ratio of modified poly-ε-lysine to poly(ethylene glycol) diacrylate polymer in the hydrogel is from 40:60 to 80:20; and from 30% to 75% of the amino groups have been functionalised with pendant functional acrylate groups. Suitably, from 30% to 60% of the amino groups have been functionalised with pendant functional acrylate groups. More suitably, from 30% to 50% of the amino groups have been functionalised with pendant functional acrylate groups.

In an embodiment, the weight ratio of modified poly-ε-lysine to poly(ethylene glycol) diacrylate polymer in the hydrogel is from 50:50 to 60:40, and from 30% to 75% of the amino groups have been functionalised with pendant functional acrylate groups. Suitably, from 30% to 60% of the amino groups have been functionalised with pendant functional acrylate groups. More suitably, from 30% to 50% of the amino groups have been functionalised with pendant functional acrylate groups.

In an embodiment, the weight ratio of modified poly-ε-lysine to poly(ethylene glycol) diacrylate polymer in the hydrogel is from 40:60 to 80:20; and from 30% to 75% of the amino groups have been functionalised with pendant functional acrylate groups. Suitably, from 30% to 60% of the amino groups have been functionalised with pendant functional acrylate groups. More suitably, from 30% to 50% of the amino groups have been functionalised with pendant functional acrylate groups.

In an embodiment, the weight ratio of modified poly-ε-lysine to poly(ethylene glycol) diacrylate polymer in the hydrogel is from 50:50 to 60:40, and from 30% to 75% of the amino groups have been functionalised with pendant functional acrylate groups.

In certain embodiments, the gel forming component of a hydrogel comprising a crosslinked polymer in the hydrogel blend is a blend of modified poly-ε-lysine and poly(ethylene glycol) diacrylate. Suitably, the modified poly-ε-lysine is the only gel forming component in the hydrogel.

In certain embodiments, the gel forming component of a hydrogel comprising a crosslinked polymer in the hydrogel blend is the modified poly-ε-lysine. Suitably, the modified poly-ε-lysine and poly(ethylene glycol) polymer are the only gel forming components in the hydrogel.

### Modified Poly-ε-Lysine Polymers

As described herein, the invention provides a modified poly-ε-lysine polymer, in which 10% to 75% of the amino groups are functionalised with pendant functional acrylate groups. Such polymers can be used in the formation of the hydrogels of the present invention.

Thus, in an aspect, there is provided a modified poly-ε-lysine polymer comprising a repeating monomer unit according to formula (A) below:

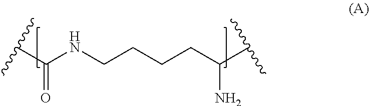

wherein from 10% to 75% of the amino groups have been functionalised with pendant functional acrylate groups.

Suitably, the functional acrylate groups have the structure:

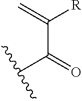

wherein R is selected from hydrogen or (1-4C)alkyl. Suitably, R is hydrogen or methyl

Thus, from 10% to 75% of the repeating units in the modified poly-ε-lysine polymer will have a formula (A′)

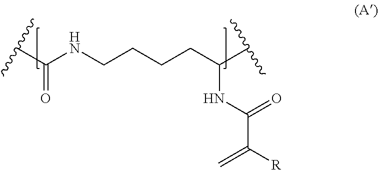

wherein R is H or (1-4C)alkyl;

and the remaining repeating units will have the formula (A) below:

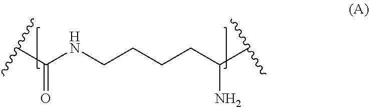

Suitably, R is selected from H, methyl or ethyl. More suitably R is H or methyl. Suitably therefore the pendant acrylate groups are selected from acrylate or methacrylate. Thus, from 10% to 75% of the repeating units in the modified poly-ε-lysine polymer may have a formula of either (A″) or (A″):

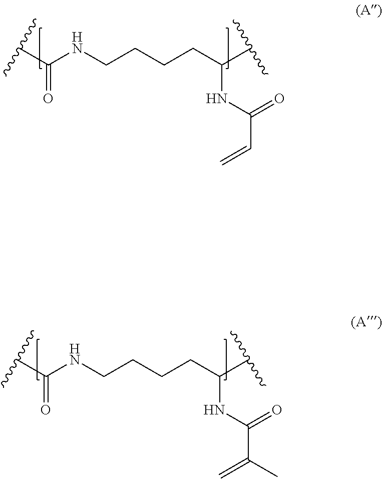

and the rest will have the formula (A).

Suitably, from 20% to 60% of the amino groups in the modified poly-ε-lysine are functionalised with pendant functional acrylate groups, more suitably from 20% to 50% of the amino groups are functionalised with pendant functional acrylate groups.

### Method of Making Modified Poly-ε-Lysine Polymers

As previously stated, the present invention also provides a method of preparing a modified poly-ε-lysine, the method comprising reacting poly-ε-lysine with an acrylic acid derivative or an acrylic acid anhydride derivative.

Suitably, the acrylic acid derivative or an acrylic acid anhydride derivative is selected from acrylic acid, methacrylic acid, acrylic anhydride or methacrylic anhydride. Suitably, the acrylic acid derivative or an acrylic acid anhydride derivative is methacrylic anhydride.

Suitably, the acrylic acid derivative or an acrylic acid anhydride derivative is added at a molar ratio of from 0.1:1 to 0.75:1, with respect to the molar content of amino functions available along the pεK backbone. More suitably, the acrylic acid derivative or an acrylic acid anhydride derivative is added at a molar ratio of from 0.3:1 to 0.6:1, with respect to the molar content of amino functions available along the pεK backbone.

Suitably, the acrylic acid derivative or an acrylic acid anhydride derivative is added at a molar ratio of from 0.05:1 to 0.75:1, with respect to the molar content of amino functions available along the pεK backbone. More suitably, the acrylic acid derivative or an acrylic acid anhydride derivative is added at a molar ratio of from 0.25:1 to 0.6:1, with respect to the molar content of amino functions available along the pεK backbone. More suitably, the acrylic acid derivative or an acrylic acid anhydride derivative is added at a molar ratio of from 0.25:1 to 0.4:1, with respect to the molar content of amino functions available along the pεk backbone.

Suitably, the modified poly-ε-lysine has a molecular weight of from 2000 to 8000 Da, more suitably from 3000 to 7000 Da, most suitably from 4000 to 6000 Da.

Suitably, the modified poly-ε-lysine has a degree of polymerisation of from 10 to 35, for example 15 to 30 or 15 to 25.

Suitably, the modified poly-ε-lysine has a degree of polymerisation of from 25 to 35.

### Therapeutic Uses and Applications

The hydrogels of the present invention find particular use in the treatment of disease or damage to the corneal epithelium. The hydrogels of the present invention may be the form of a bandage contact lens when used in such treatments.

The hydrogels of the present invention also find particular use in the treatment of disease or damage to the corneal endothelium. The hydrogels of the present invention may be used as a biomaterial substrate for cell growth, which can be used in the treatment of disease of damage to the corneal endothelium. The hydrogels of the present invention may be the form of a support (i.e. a construct or scaffold) on which to grow cells.

Thus, there is provided a hydrogel or a bandage contact lens as defined herein, for use in the treatment of damage to the corneal epithelium or corneal endothelium. Such treatment may involve aiding the healing of the corneal epithelium or corneal endothelium.

The hydrogels of the present invention may be used in the treatment of any corneal epithelial defect (for example persistent corneal defects, microbial keratitis, damage following corneal surgery or in post corneal cross-linking for keratoconus) or ocular epithelial dysfunction (for example limbal stem cell deficiency, microbial keratitis). Such treatment may aid the healing process and help to repair such defects. The treatment may also involve reducing the discomfort or pain caused by these conditions.

The hydrogels of the present invention may be used in the treatment of any corneal endothelial dysfunction, for example in the treatment of corneal swelling due corneal oedema (e.g. bullous keratopathy, Fuchs Endothelial Dystrophy, Congenital Hereditary Endothelial Dystrophy, hydrops of the cornea in keratoconus). The treatment may also involve reducing the discomfort or pain caused by these conditions.

The hydrogels of the present invention may also be used to correct refractive error.

The hydrogels of the present invention may also be used to reduce degradation of an amniotic membrane transplant, to reduce dryness of an amniotic membrane transplant or other topical ocular treatment.

The hydrogels of the present invention may also be used for cosmetic purposes.

The hydrogels of the present invention may be used on their own as the sole therapy. Alternatively, the hydrogels may be administered as part of a combination therapy with one or more other eye treatments. Such conjoint treatment may be achieved by way of the simultaneous, sequential or separate administration of the individual components of the treatment.

By way of example, collagenic eye disorders may result in a number of other undesirable symptoms to the patient, such as, for example, pain, infection, dryness and discomfort. Accordingly, the hydrogels of the present invention may be used in combination with one or more additional medicaments or additives, such as, for example, hydrating agents, antibiotics, steroids, anaesthetics and antihistamines.

### Support for Cell Growth

The hydrogels of the present invention also find use in ex vivo cell expansion for ocular tissue engineering, as a vehicle to support implantation of said cells, and to support the growth and function of said cells in vivo. Thus, the hydrogels may be used as a biomaterial substrate to act as a support (i.e. a construct or scaffold) for cell growth e.g. in tissue engineering applications. The present invention provides a hydrogel as defined herein, for use as a scaffold for corneal endothelial cells (e.g. as a corneal endothelial graft material) or corneal epithelial cells.

The hydrogels of the present invention may also be used as an alternative to a corneal transplant. For example the hydrogels may be used as an insert to support a damaged/diseased/affected cornea, or as a transplant to replace part of the damaged cornea. The hydrogels may also be used as a stromal replacement in the treatment of keratoconus. The hydrogels may also be used in re-shaping of the cornea, for example reshaping a donor cornea for transplantation. The hydrogels may also be used to stabilise corneal shape, for example following refractive surgical procedures.

Transparent and non-transparent hydrogels of the present invention may also find use in conjunctival epithelial repair, in retinal pigment epithelial cell and photoreceptor transplantation and in ex vivo cell expansion for ocular tissue engineering. Thus, it is not essential that the hydrogels of the present invention are transparent.

### Ex Vivo Cell Expansion for Ocular Tissue Engineering:

Cell based therapies are being developed for many ocular diseases including diabetic retinopathy, age related macular degeneration, glaucoma and corneal and surface ocular diseases. For many of these the relevant cell type needs to be harvested and expanded in culture prior to use in the patient. There is a critical need to develop substrates with tailored surface properties that fulfil GMP conditions and in particular reduce or eliminate the need for serum within the culture environment. The peK hydrogels of the present invention can be used to tailor these surfaces to mimic protein containing substrates that promote cell adhesion and growth.

The hydrogels of the present invention may be modified to include cell adhesive proteins to increase the adhesion of cells. Such cell adhesive proteins may be bound to the surface by covalent bonds or via electrostatic interactions. However, due to the presence of free amino groups which can bind some cells, the presence of cell adhesive proteins may not be required.

### Conjunctival Epithelial Repair:

The conjunctiva plays key roles in providing protection to the eye and maintaining homeostasis of its ocular surface. Multiple diseases can impair conjunctival function leading to severe consequences that require surgical intervention. Small conjunctival defects can be repaired relatively easily, but larger defects rely on tissue grafts which generally do not provide adequate healing. A tissue engineering approach involving a biomaterial substrate capable of supporting a stratified conjunctival epithelium with embedded mucin-secreting goblet cells offers a potential solution.

Conjunctival diseases are numerous, but of greatest interest to tissue engineering are those that result in significant loss of healthy conjunctiva, which may in turn cause corneal problems with loss of vision, or double vision. These include mucous membrane pemphigoid, Stevens-Johnson syndrome, trauma including burns, malignancy, and complicated glaucoma or socket surgery. Of these, malignancy potentially offers the greatest scope for engineered conjunctival substitutes as following complete resection of a tumour there may be an extensive defect, but the remaining tissue is healthy and provides a good host environment for a transplant.

Thus, the hydrogels of the present invention may therefore be used as a support for a stratified conjunctival epithelium with embedded mucin-secreting goblet cells.

### Retinal Pigment Epithelial Cell and Photoreceptor Transplantation

Age-related macular degeneration (AMD) is the most common cause of blindness in people over 65. Current treatment options are expensive and at best only limit disease progression. Dysfunction and death of the pigmented cell layer under the light sensitive retina (retinal pigment epithelium [RPE]) is pivotal to the development and progression of AMD. The peK hydrogels of the present invention could be used to provide a substrate on which to grow a monolayer of RPE cells and transplant these into the back of the eye to replace the damaged RPE layer. There is also potential to use the peK hydrogels of the present invention as a substrate to build a complex cellular transplant comprising the RPE and overlying photoreceptors.

### DETAILED DESCRIPTION OF THE DRAWINGS

The present invention is further defined with reference to the accompanying figures, described below:

FIG. 1: Proposed mechanism of reaction of pεK with methacrylic anhydride.

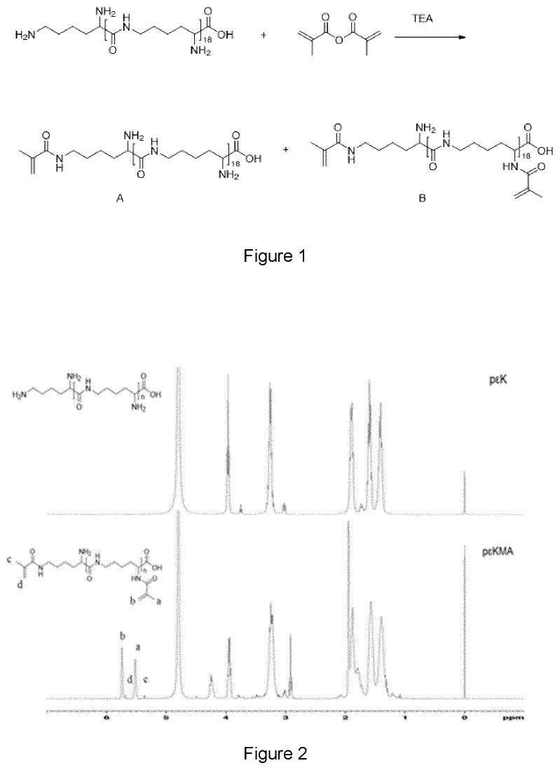

FIG. 2: ¹H NMR spectra of pεK and pεKMA-L recorded in D2O at room temperature. The additional peaks (a, b, c and d) show the covalent binding of methacrylated groups to pεK backbone.

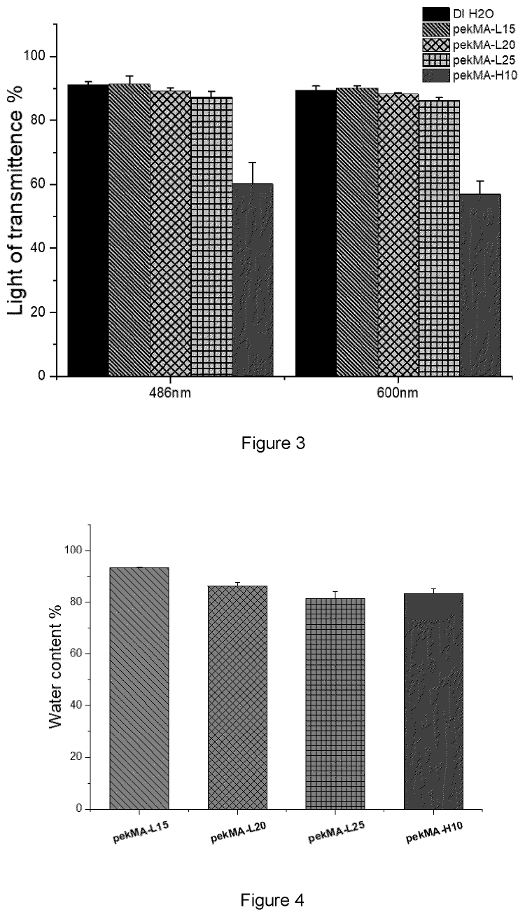

FIG. 3: Quantification of transparency on low and high DOF pεKMA hydrogels compared to DI H₂O (n=6).

FIG. 4: Comparison of water content between low and high DOF pεKMA hydrogels (n=6).

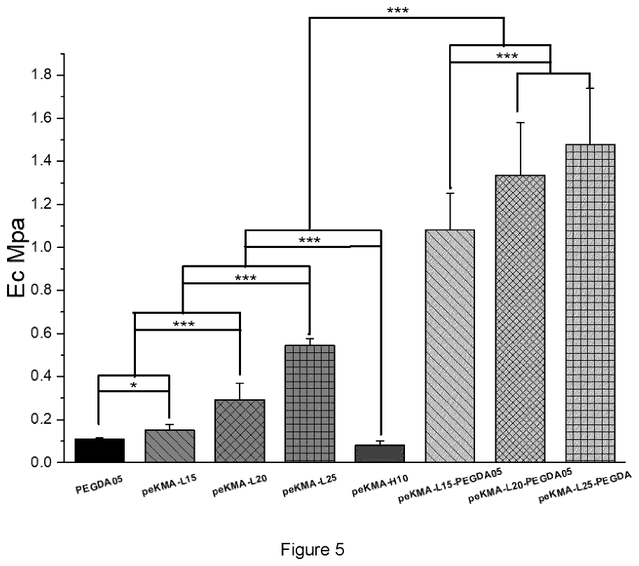

FIG. 5: Compressive modulus (Ec) of pεKMA, PEGDA and both with low and high DOF (n=3). Data are presented as mean±SD. ***: p<0.001 **: p<0.01, *: p<0.05.

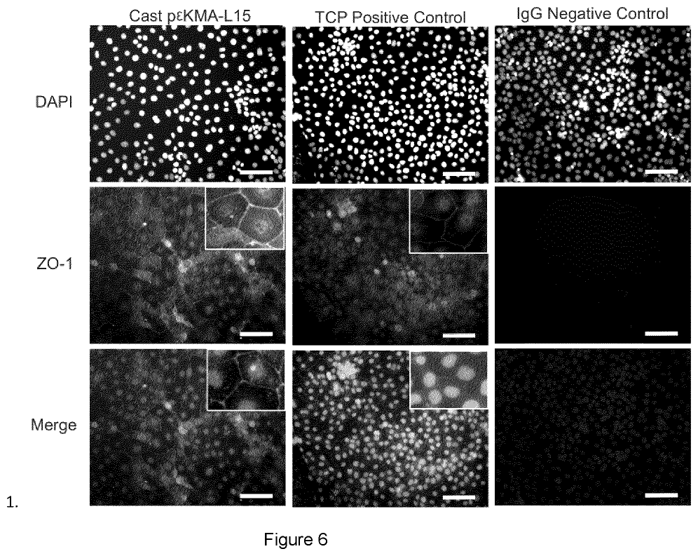

FIG. 6: Typical DAPI and ZO-1 staining of HCE-T cells after 3 weeks seeded on cast pεKMA-L15. Imaged on a Nikon Ti-E live-cell fluorescent microscope. Scale bars represent 100 μm

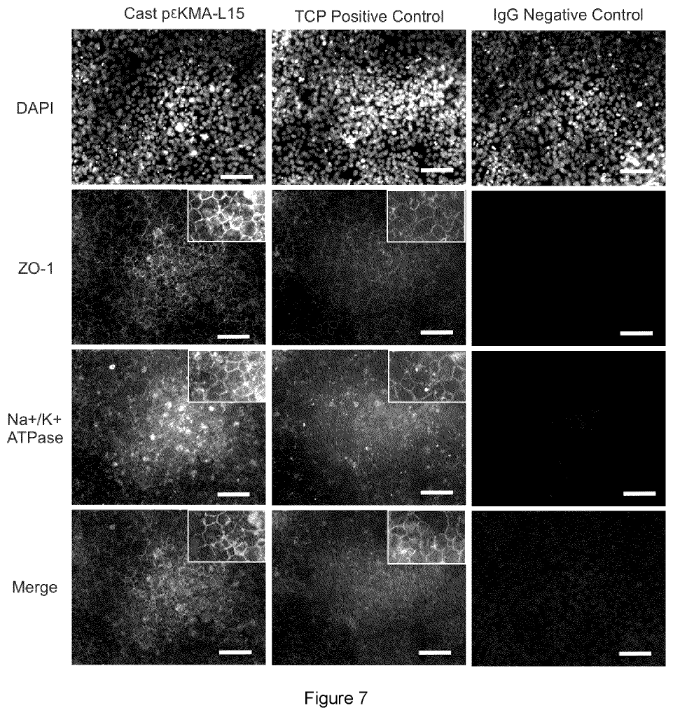

FIG. 7: Typical DAPI, ZO-1 and Na⁺/K⁺/ATPase staining of HCEC-12 cells after 3 weeks seeded on cast and 3D printed pεKMA-L15. Imaged on a Nikon Ti-E live-cell fluorescent microscope. Scale bars represent 100 μm.

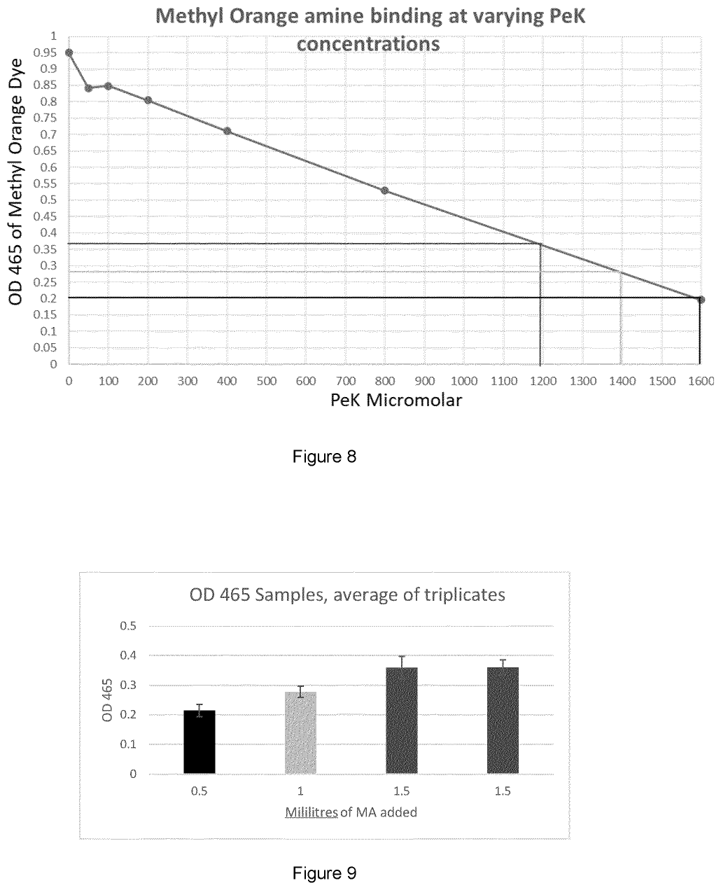

FIGS. 8 and 9: Results of determination of amine groups remaining using methyl orange. FIG. 8 shows the optical density of methyl orange at 465 nm versus the pεK concentration. FIG. 9 shows the optical density of methyl orange at 465 nm versus the millilitres of methacrylic anhydride added.

### EXAMPLES

The inventors have developed a poly-ε-lysine based hydrogel with excellent optical and mechanical properties that can be controlled by the nature and percentage of the cross-links and the density of the peptide. The inventors have demonstrated that primary CECs adhere and proliferate on the surface of the gel to produce a confluent monolayer and that this graft has the physical integrity to be manipulated for transplantation using a clinical delivery device.

### Photo Crosslinked Poly-ε-Lysine Hydrogel for Cornea Repair

### Materials

Poly-ε-lysine (pεK) (Epsiliseen®-H, ˜5000 Da) was purchased from Siveele B.V. Methacrylic anhydride (MA), poly(ethylene glycol) diacrylate (PEGDA, Mn=700 Da) and triethylamine (TEA) were purchased from Sigma-Aldrich. 2-Hydroxy-1-[4-(2-hydroxyethoxy)phenyl]-2-methylpropan-1-one (12959) was purchased from Fluorochem Limited (Glossop, UK). All other chemicals were purchased from Sigma Aldrich unless specified.

### Methods

Synthesis of Methacrylated (pεK MA)

pεKMA was dissolved (10 wt.-%) in DI H₂O and stirred at room temperature until complete dissolution was obtained. 0.1 M NaOH solution was added to achieve a pH of 7.4. MA and TEA were added at molar ratios of 0.3:1 (“low”) and 0.6:1 (“high”) with respect to the molar content of amino functions available along the pεK backbone. After 24 hours the reaction mixture was precipitated in 10-volume excess of pure acetone for 8 hours. The precipitation was dissolved in 100 mL of DI H₂O and filtered, following by dialysis (MWCO 1000 Da) against DI H₂O for three days. Finally, the purified solution was filtered and lyophilised. TNBS and ¹H NMR (400 MHz Bruker) was used to determine the structure and composition of pεKMA.

Preparation of UV-Induced pεKMA Hydrogel

pεKMA were dissolved (10 to 25 wt. %) in 0.01M PBS containing 1 wt. % 2-hydroxy-40-(2-hydroxyethoxy)-2-methylpropiophenone (12959) photoinitiator. The solution was centrifuged at 3000 rpm for 5 minutes to remove any air bubbles, followed by casting (600 mL well⁻¹) onto a 24-well plate (Corning Costar) and UV curing (Spectroline, 346 nm, 8 mW cm²) for 10 min from each side of the dish (light-sample distance: 10 cm). The formed hydrogels were rinsed with distilled water, dehydrated via an ascending series of ethanol solutions and air dried.

(2,4,6)-Trinitrobenzenesulfonic acid (TNBS)

The degree of pεK methacrylation was determined via the TNBS assay (n=3). Briefly, 10 mg of dry pεKMA samples was mixed with 1 mL of 4 wt % NaHCO₃ (pH=8.5) and 1 mL of 0.5 wt % TNBS solution. The mixture was placed on a rocker with mild shaking at 40° C. for 4 hours and 3 mL of 6 M HCL solution was added and the mixture was incubated at 60° C. for another hour. The solution was allowed to cool to room temperature, followed by mixing with 5 ml distilled water, and extracted three times with diethyl ether to remove any unreacted TNBS reagent. All samples were read against blank using UV-Vis spectrophotometer (SPECTROstar Nano, BMG Labtech, Germany) at 346 nm, and the content of unfunctionalized amino groups and the degree of functionalisation (DOF were calculated from the following equations (eq.1 and eq.2):

$$\frac{mol ( p ε kMA )}{g ( p ε kMA )} = \frac{2 \times Abs ( 346 nm ) \times 0.02}{1.46 \times {10}^{4} \times b \times x} \qquad ( Eq . 1 )$$

Where Abs (346 nm) is the UV absorbance value recorded at 346 nm, 2 is the dilution factor, 0.02 is the volume of the sample solution in litres, 1.46×10⁴ is the molar absorption coefficient for 2,4,6-trinitrophenyl lysine (in M⁻¹ cm⁻¹), b is the cell path length (1 cm), and x is the weight of the dry sample. DOF was calculated via Eq.2

$$DOF = 100 - \frac{mol ( p ε kMA )}{mol ( p ε K )} \times 100 \qquad ( Eq . 2 )$$

### Quantification of Water Content

The water content was measured to investigate the overall portion of the covalent hydrogel network in distilled water. Dry methacrylated pεK hydrogel disks (ma: 0.02 to 0.03 g) were equilibrated in distilled water over night and the resulting samples were recorded as mw. The water content (WC) was calculated by the following equation:

$$WC = \frac{mw - md}{mw} \times 100 \qquad ( Eq . 3 )$$

### Compression and Transparency Test

The quantification of compressive properties of the methacrylated pεK hydrogel were performed using Univert mechanical tester (CellScale, US). Swollen pεK hydrogel disks (φ: 15.6 mm; h: 6-10 mm) were compressed at room temperature at the rate of 5 mm*min⁻¹. A 10 N load cell was used to generate the stress-strain curve and the compression modulus was quantified as the slope of the linear region of the plot at 25-30% strain. Three replicates were employed for both low (0.3:1, with the aim of achieving ˜30% functionalisation) and high (0.6:1, with the aim of achieving 60% functionalisation) pεKMA hydrogels.

The visible light transmittance of the pεKMA hydrogels was evaluated by applying a UV-vis spectrophotometer (SPECTROstar Nano, BMG labtech, Germany) from 400 to 600 nm.

### ¹H NMR

¹H NMR spectra of pεK and pεKMA were analysed on a Bruker AVANCE III 400 MHz. Thirty-two scans were acquired for signal to noise averaging, and a recycle delay of 30 s was used to ensure quantitative spectra. In all cases D₂O was used as the solvent and a concentration of 10 mg/mL was used for all samples.

### Cell Culture

Human corneal epithelial cells (HCE-T) were cultured in Dulbecco's modified Eagle's medium nutrient F-12 (DMEM/F12), supplemented with 10% fetal bovine serum (FBS), and 2.5 mg mL⁻¹ penicillin-streptomycin, in a humidified incubator at 37° C. and 5% CO₂. Human corneal endothelial cells (HCEC-12) were cultured in Medium 199 with Hams F12 Nutrient mix at a 1:1 ratio, supplemented with 5% FBS, 2.5 mg mL-1 Amphotericin B and 2.5 mg mL-1 penicillin-streptomycin. Cells were passaged every 3 days with 0.25% trypsin/0.02% EDTA. Freshly-synthesised, UV-cured pεKMA samples were incubated in a 70 vol % ethanol solution under UV light. Retrieved samples were washed in PBS three times to avoid any remaining ethanol, prior to cell seeding.

Immunocytochemistry was used to determine HCE-T tissue distribution of ZO-1 and HCEC-12 distribution of ZO-1 and Na⁺/K⁺ ATPase. Briefly, before cell seeding, the hydrogels were sterilized in 70% ethanol for 1 hour, then washed with PBS and left overnight. Both cell types were seeded separately at a density of 2.5×10⁴ cm⁻² and left to culture for 3 weeks. After 3 weeks, the cells on pεKMA hydrogels were fixed using 10% (v/v) neutral buffered formalin (NBF) (Sigma-Aldrich, UK) for 15 minutes. The cells were washed with PBS before adding 1% (v/v) Triton X-100 (Sigma-Aldrich, UK) to permeablise the cells. After 10 minutes, the Triton-X was removed and the cells again washed with PBS. 10% (v/v) Goat serum (GS) in 1% (v/v) BSA (both Sigma-Aldrich, UK) was used to block non-specific binding sites for 30 minutes at 37° C. Primary antibodies zonula occludens 1 (ZO-1) (Invitrogen Rabbit polyclonal 61-7300, Thermo Fisher, UK) and Na+/K+-ATPase a (Mouse monoclonal IgG1, sc-58628, Insight Biotechnology Limited, Middlesex, UK) and IgG controls (all 2.5 μg cm⁻³) were made up in 1% BSA. ZO-1 primary antibody was added to the fixed HCE-Ts and HCEC-12s, and Na+/K+-ATPase antibody added to HCEC-12s and the cells were left at 4° C. overnight. The primary antibody was removed the following morning, and the cells washed with PBS-Tween (PBS containing 0.1% (v/v) Tween-20) (Sigma-Aldrich, UK). Secondary antibodies Alexa Fluor 488 Goat anti-Rabbit (Invitrogen A-11008, Thermo Fisher, UK) and Alexa Fluor 594 Goat anti-Mouse (Invitrogen A-32742, Thermo Fisher, UK) were added to the cells and the plates left at 37° C. for 1 hour in the dark. Again, the cells were washed with PBS-Tween. DAPI was used to stain the nuclei of the cells for 30 minutes at room temperature. After a final PBS wash, the hydrogels were imaged using a Nikon Ti-E live-cell fluorescent microscope and Nikon Elements software (both Nikon Instruments UK, Surrey, UK).

### Statistical Analysis

Data are shown as mean±standard deviation (SD). Statistical analysis was carried out with the Student's t-test. A p value lower than 0.05 was considered to be significant.

Preparation of peKMA/PEGDA Blends

For a typical peKMA05-PEGMA05 hydrogel, pεKMA (5 wt. %) and PEGDA (5 wt. %) were dissolved in 0.01M PBS containing 1 wt. % 2-hydroxy-40-(2-hydroxyethoxy)-2-methylpropiophenone (I2959) photoinitiator. The solution was centrifuged at 3000 rpm for 5 minutes to remove any air bubbles, followed by casting (600 mL well 1) onto a 24-well plate (Corning Costar) and UV curing (Spectroline, 346 nm, 8 mW cm⁻²) for 10 min at each side of the dish (light-sample distance: 10 cm). The formed hydrogels were rinsed with distilled water, dehydrated via an ascending series of ethanol solutions and air dried.

### Results

The nomenclature for samples is used as follows: functionalised methacrylated pεK samples are coded as pεKMA-L or pεKMA-H, where L and H describes the low (0.3:1) and high (0.6:1) methacrylic anhydride/pεK molar ratio used in the reaction; UV-induced hydrogel are coded as pεKMA-LXX and pεKMA-HXX, where L and H have the same meaning previously described and XX means the weight percentage of the pεKMA adducts for the gel formation, i.e. pεKMA-L15, pεKMA hydrogel was made with a fixed concentration of 15 wt % with low degree functionalised pεKMA adducts.

Derivatization of pεK with Photo-Curable Methacrylic Groups.

The reaction of pεK with methacrylamide adducts leads to consumptions of free amino groups and TNBS, a colorimetric assay was used to quantify the molar content of free amino groups in both modified and unmodified pεK and the degree of functionalisation (DOF) was indirectly quantified. An overall content of free amino groups of 6.7*10⁻³ mol*g⁻¹ was measured on native unmodified pεK via TNBS assay, the range of high and low molar ratio selected for the modification proved to be directly affecting the degree of functionalisation (DOF: pεKMA-L=35.07±6.20% and pεKMA-H=71.6±2.15%).

TABLE 1
TNBS assay quantification of the amino group molar
content and degree of functionalisation (DOF) in pεk and
pεKMA with high and low modification (n = 2).

| | Amine groups/mol*g⁻¹ (×10⁻⁴) | DOF (%) |
|---|---|---|
| pεK | 6.70 ± 1.30 | n/a |
| pεKMA-L | 4.35 ± 0.49 | 35.07 ± 6.20 |
| pεKMA-H | 1.90 ± 0.28 | 71.6 ± 2.15 |

n/a: not applicable

¹H NMR spectra of both pεK and pεKMA confirms the successful functionalisation of methacrylic groups (FIG. 2). The average molecular weight of pεK was calculated from FIG. 1A to be 3000. By comparing the ¹H NMR spectra of pεK and pεKMA, the addition of the peak signals at 5.38 and 5.65 ppm, 5.51 and 5.72 ppm can be attributed to the double bond of the methacrylate group confirming the successful methacrylation. The degree of functionalisation for pεKMA-L was 38% from the ratio of the peaks a and b, c and d, which is in line with the TNBS results.

### Transparency

Transparency studies were conducted at the room temperature measuring the light transmittance (FIG. 3). By comparing to DI H₂O, all pεKMA-L samples exhibit over 85% transparency, pεKMA-L15: 91±3, pεKMA-L20: 89±1, pεKMA-L25: 87±2% at 386 nm; 90±1, 88±1 and 86±1 at 600 nm respectively. As expected, high DOF pεKMA shows a significant lower transparency 60±7 (386 nm) and 57±4% (600 nm). The results suggested that pεKMA hydrogel at a low concentration had the highest transparency with a transmittance value over than 90% in both 386 and 600 nm at room temperature.

### Water Content

All UV-cured networks displayed an average water content of at least 80% (FIG. 4). Sample pεKMA-L15 showed the highest water content 93±1%, followed by pεKMA-L20 (86±1%), pεKMA-H10 (83±2%) and pεKMA-L20 (81±3%).

### Compression Testing

The compressive module (FIG. 5) of Poly(ethylene glycol) diacrylate (PEGDA05) (Ec=0.08±0.02 MPa), was similar to pεKMA-H10 (Ec=0.11±0.01 MPa). The compressive modulus of the pεKMA-L increased with increase in wt % of the pεKMA with pεKMA-L15 (Ec=0.15±0.03 MPa), pεKMA-L20 (Ec=0.30±0.07 MPa) and pεKMA-L25 the highest (Ec=0.55±0.03 MPa). The compressive modulus of all pεKMA-L samples were significantly enhanced when blend with PEGDA05, a nearly seven-fold increase was measured on pεKMA-L15-PEGDA05 (Ec-1.09±0.17 MPa), four-fold increase on pεKMA-L20-PEGDA05 (Ec-1.33±0.25 MPa) and three-fold increase on pεKMA-L25-PEGDA05 (Ec=1.47±0.26 MPa).

### In Vitro Cell Cyto-Compatibility

HCE-Ts were cultured on pεKMA-L15 for 3 weeks and in this time formed a complete monolayer across the hydrogel surfaces (FIG. 6). Positive ZO-1 staining demonstrates tight junction formation in between HCE-T cells on the pεKMA hydrogel, a typical characteristic of epithelial tissue. The cells look slightly less confluent on the hydrogels compared to TCP, however, the ZO-1 staining is comparable with the positive control. Immunostaining gives confidence of cyto-compatibility with a corneal epithelial cell line.

HCEC-12s seeded onto the pεKMAL-15 showed positive staining for ZO-1 and Na⁺/K⁺/ATPase antibodies (FIG. 7). The HCEC-12s were fixed on the pεKMA substrates after 3 weeks of culture. Positive ZO-1 staining demonstrates the formation of a monolayer after 3 weeks on the pεKMA, which is comparable with the TCP positive control. Further positive staining of Na⁺/K⁺/ATPase demonstrates the monolayer is functional due to the presence of Na⁺/K⁺/ATPase pumps in the plasma membrane of the endothelial cells. Again, the positive staining is similar across pεKMA and TCP. The HCEC-12s display a cobblestone morphology compared to the HCE-Ts, which is typical of human corneal endothelial cells. These findings suggest the cyto-compatibility of pεKMA with this cell line.

Additional Synthesis of Methacrylated Poly-ε-Lysine (pεKMA)

### Materials

Poly-ε-lysine (pεK) (Epsiliseen®-H, ˜5000 Da) was purchased from Siveele B.V. Methacrylic anhydride (MA, Catalogue ref. 276685), Triethylamine (TEA, Catalogue ref. 471283), N,N-Dimethylformamide (DMF, Catalogue ref. 227056-1L) were purchased from Merck (Sigma-Aldrich). Acetone AR 2.5L (Catalogue ref. 10252232, discounted price is £5.75) was purchased from Fisher Scientific. Deionised water and Phosphate buffer saline (PBS). Aqueous solution sodium hydroxide NaOH (0.5 M). Liquid nitrogen collected in a cylindrical Dewar.

### Procedure for Synthesis

1. Clamp the round bottom flask on the retort stand with magnetic stirrer plate beneath the flask.
2. Weight 10 g of pεK using a weighing boat.
3. Carefully transfer pεK into the round bottom flask.
4. Add 50 mL of deionised/ultrapure water, add a stirring bar and continue stirring until fully dissolved at room temperature (20° C.).
5. Adjust pH to 7 by adding 0.5 M NaOH.
6. Add 1 mL of DMF to the pεK solution using 1 ml syringe
7. Add 1.36 mL of TEA to solution in (6) while stirring
8. Add 1.5 mL of MA dropwise to solution in (7) over a period of 1 h with continuous stirring.
9. Allow the reaction to proceed for 12 h at room temperature.
10. Terminate reaction by adding 10 mL of DPBS.
11. Precipitate pεKMA with 10-volume (˜600 mL) excess of acetone for 8 h.
12. Carefully decant the liquid but retain the solid (sometimes it could be viscous (honey-like) precipitate.
13. Reconstitute the precipitate in deionised water (100 mL).
14. Transfer pεKMA into dialysis tubing (MWCO 0.5-1 kDa) and dialyse against ultrapure water for 3 days. Replace the water every 6 h.
15. Transfer solutions into 50 mL Falcon tubes and freeze by immersion in liquid nitrogen.
16. Freeze-dry for 1 week to obtain pure pεKMA powder.
(Step (8) variation: add 0.5 ml, 1 ml and 1.5 ml, corresponding to a MA molar ratio of 0.085:1, 0.170:1 and 0.255:1, respectively, with respect to the molar content of amino functions available along the pεK backbone)

### Methyl Orange Amine Group Determination:

An alternative method of functionalised amine quantification using methyl orange dye was adapted from the original protocol (Itzhaki, 1972). A standard curve of methyl orange concentration against absorbance was composed using known pεK concentrations as standards. A 2 mM methyl orange solution was made up by dissolving it (32.7 mg) in H₂O (50 cm³). A 1.6 mM pεK stock solution was also made up by dissolving it (13.2 mg) in H₂O (50 cm³). This was then serial diluted to 800, 400, 200, 100 and 50 UM concentrations with water. The methyl orange stock (500 mm³) was added to each of the pek concentrations (500 mm³) in a 1.5 cm³ centrifuge tube. These were then mixed by adding them to a 50 cm³ Falcon tube and placing them on a roller for 30 min. Each 1.5 cm³ tube was then centrifuged with a Heraeus Biofuge Pico at 5345×g (Thermo Scientific, Waltham, WA, USA) for 5 min. The tubes were retrieved and an aliquot of the supernatant (150 mm³) removed and added to H₂O (2.85 cm³) in a cuvette. The cuvette was inverted 3 times before measuring the absorbance at 465 nm using a Cecil CE292 UV spectrophotometer (Cecil Instruments Ltd., Cambridge, England) against a water blank. A standard curve was then compiled and used to calculate the amount of amino groups in a sample from the concentration of methyl orange. Each unknown sample was prepared in the same way as for the standards above.

### Results

Three PEK variants (by adding 1.5, 1.0 or 0.5 ml of MA (step 8) resulted in 33%, 19% and 5% MA functionalisation respectively. All three variants produced gels following exposure to UV. Variant 0.5 was very weak and was not easy to handle. The other two variants produced handleable gels but variant 1.0 was weaker than variant 1.5. Results are shown in Tables 2 and 3 and in FIGS. 8 and 9.

TABLE 2
Methacrylation level at different levels
of added methacrylic anhydride

| Methacrylic | | | |
|---|---|---|---|
| Anhydride | Optical Density | | |
| (MA) added | at 465 nm of | | Methacrylation |
| (ml) | PEKMA samples | % Amine Remaining | Level |
| 0.5 ml | 0.215 | 95% | 5% |
| 1 ml | 0.278 | 81% | 19% |
| 1.5 ml | 0.359 | 67% | 33% |
| 1.5 ml | 0.360 | 66% | 34% |
| (Repeat) | | | |

TABLE 3
Hydrogel formation at different levels of methacrylic anhydride

| MA added | Hydrogel formation |
|---|---|
| 0.5 ml | Yes (Weak gel) |
| 1 ml | Yes |
| 1.5 ml | Yes |

### Calculating Molar Ratio of MA to Molar Content of Primary Amino Group on Poly-ε-Lysine

Assuming the molecular weight of pεK=5000 g/mol (Da)
Molecular weight of methacrylic anhydride (MA)=154.165 g/mol
Density of MA=1.035 g/ml
Calculate molar content of amino group;
1 mole of pεK=5000 g/mol
Mass of amino group (NH₂) in 5000 g of pεK is 18 g/mol×20=360 g/mol. (Note: molar mass of NH₂=18 g/mol; assuming there are 20 amino groups).
Then in 1 g of pεK, mass of NH₂=360×1/5000=0.072 g.
Hence, if 10 g of pεK was used for the synthesis of pεKMA, then mass of NH₂ in the 10 g=0.072×10=0.72 g.
Amount of NH₂ in moles/10 g of pεK=0.72/18=40 mmol
Calculate molar content of MA;
Mw of MA=154.165 g/mol
Density=mass/volume; mass of MA=density×volume used
For 0.5 mL, mass=1.035×0.5=0.5175 g
Amount of MA in mole/0.5 mL=0.5175/154.165=3.4 mmol
For 1.0 mL, amount of MA=6.8 mmol.
For 1.5 mL, amount of MA=10.2 mmol
Calculate molar ratio of MA to molar content of amino groups;
When 0.5 mL MA is used, then the molar ratio of MA to amino group on pεK=0.085:1.
When 1.0 mL MA is used, then the molar ratio of MA to amino group on pεK=0.170:1.
When 1.5 mL MA is used, then the molar ratio of MA to amino group on pεK=0.255:

### Numbered Paragraphs

The following numbered paragraphs define particular aspects and embodiments of the invention.
1. A method for making a hydrogel, the method comprising:
(i) providing a solution comprising a modified poly-ε-lysine polymer which comprises a repeating monomer unit according to formula (A) below:

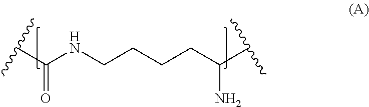

wherein from 10% to 75% of the amino groups have been functionalised with pendant functional acrylate groups; and
ii) exposing the modified poly-ε-lysine polymer to UV light to crosslink the modified poly-ε-lysine and provide the hydrogel.
2. The method according to paragraph 1, wherein from 20% to 60%, preferably 20% to 50%, of the amino groups have been functionalised with pendant functional acrylate groups.
3. The method according to paragraph 1 or paragraph 2, wherein the functional acrylate groups have the following structure:

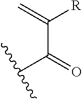

wherein R is selected from H or (1-4C)alkyl.
4. The method according to any one of the preceding paragraphs, wherein the pendent acrylate groups are selected from acrylate or methacrylate.
5. The method according to any one of the preceding paragraphs, wherein the solution is an aqueous solution.
6. The method according to any one of the preceding paragraphs, wherein the solution comprises modified poly-ε-lysine in an amount of from 5 to 35 wt. %, optionally from 10 to 25 wt. %.
7. The method according any one of the preceding paragraphs, wherein the solution has a pH in the range of 6 to 8.5, optionally 7 to 8; and/or the solution comprises a buffer to maintain the pH, optionally wherein the buffer is selected from phosphate buffered saline.
8. The method according any one of the preceding paragraphs, wherein the solution further comprises a poly(ethylene glycol) diacrylate polymer.
9. The method according any one of the preceding paragraphs, wherein the modified poly-ε-lysine is formed by reacting poly-ε-lysine with an acrylic acid derivative or an acrylic acid anhydride derivative,
   optionally the acrylic acid derivative or an acrylic acid anhydride derivative is selected from acrylic acid, methacrylic acid, acrylic anhydride or methacrylic anhydride.
10. The method according to paragraph 9, wherein the poly-ε-lysine has a molecular weight of 2000 to 8000 Da, optionally 4000 to 6000 Da.
11. The method according to any one of the preceding paragraphs, wherein the solution further comprises a photoinitiator,
   optionally wherein the photoinitiator is selected from 2-hydroxy-40-(2-hydroxyethoxy)-2-methylpropiophenone (12959), lithium phenyl-2,4,6-trimethylbenzoylphosphinate (LAP), ethyl (2,4,6-trimethylbenzoyl) phenyl phosphinate (TPO-L) or bis(2,4,6-trimethylbenzoyl)-phenylphosphineoxide (Irgacure 819).
12. The method according to any one of the preceding paragraphs, wherein the photoinitiator is present in an amount of 0.05 to 5 wt. %; preferably 0.1 to 2 wt. %, more preferably 0.5 to 1.5 wt. %.
13. The method according to any one of the preceding paragraphs, comprising casting the solution of modified poly-ε-lysine into a film prior to exposure to UV light.
14. The method according to any one of the preceding paragraphs, wherein the UV light has a wavelength of 250 to 405 nm.
15. A hydrogel obtained by, obtainable by, or directly obtained by the method of any one of the preceding paragraphs.
16. A hydrogel comprising a polymer which comprises a repeating monomer unit according to formula (A) below:

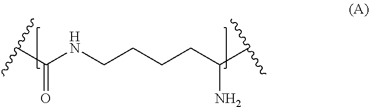

wherein from 10% to 75% of the amine groups are functionalised with an acrylate group of the formula:

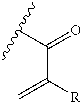

or are crosslinked by a group of the formula:

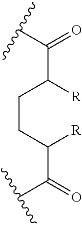

wherein each R is selected from hydrogen or (1-4C)alkyl.
17. The hydrogel according to paragraph 16, wherein each R is hydrogen or methyl.
18. The hydrogel according to any of paragraphs 15 to 17, wherein the hydrogel is in the form of a film.
19. The hydrogel according to any of paragraphs 15 to 18, wherein the hydrogel has a film thickness of from 10 to 100 microns, optionally 10 to 50 microns.
20. The hydrogel according to any of paragraphs 15 to 19, wherein the hydrogel has a water content of at least 70 wt. %.
21. The hydrogel according to any of paragraphs 15 to 20, wherein the hydrogel has a light transmittance of at least 80%.
22. The hydrogel according to any of paragraphs 16 to 21, wherein the hydrogel further comprises a poly(ethylene glycol) diacrylate polymer crosslinked to the modified poly-ε-lysine of the hydrogel.
23. The hydrogel according to paragraph 22, wherein the weight ratio of modified poly-ε-lysine to poly(ethylene glycol) polymer in the hydrogel is from 0.5:1 to 50:1, preferably from 1:1 to 40:1, more preferably from 5:1 to 25:1.
24. A bandage contact lens comprising a hydrogel according to any one of paragraphs 14 to 24.
25. A modified poly-ε-lysine polymer comprising a repeating monomer unit according to formula (A) below:

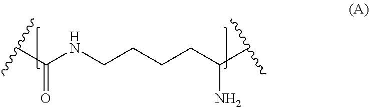

wherein from 10% to 75% of the amino groups have been functionalised with pendant functional acrylate groups.
26. A modified poly-ε-lysine polymer according to paragraph 25, wherein the pendant functional acrylate groups have the formula:

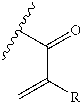

wherein R is selected from hydrogen or (1-4C)alkyl.
27. A method of preparing a modified poly-ε-lysine, the method comprising reacting poly-ε-lysine with an acrylic acid derivative or an acrylic acid anhydride derivative.
28. The method according to paragraph 27, wherein the poly-ε-lysine has a molecular weight of 2000 to 8000 Da, optionally 4000 to 6000 Da
29. The method according to paragraph 27 or 28, wherein the acrylic acid derivative or an acrylic acid anhydride derivative is selected from acrylic acid, methacrylic acid, acrylic anhydride or methacrylic anhydride.
30. A hydrogel according to any one of paragraphs 14 to 23, or a bandage contact lens according to paragraph 24, for use in therapy.
31. A hydrogel according to any one of paragraphs 14 to 23, or a bandage contact lens according to paragraph 24, for use in the treatment of disease or damage to the corneal epithelium or corneal endothelium; optionally
   a. for use in the treatment of any corneal epithelial defect (for example persistent corneal defects, microbial keratitis, damage following corneal surgery or in post corneal cross-linking for keratoconus) or ocular epithelial dysfunction (for example limbal stem cell deficiency, microbial keratitis); or
   b. for use in the treatment of any corneal endothelial dysfunction, for example in the treatment of corneal swelling due corneal oedema (e.g. bullous keratopathy, Fuchs Endothelial Dystrophy, Congenital Hereditary Endothelial Dystrophy, hydrops of the cornea in keratoconus).
32. A hydrogel according to any one of paragraphs 14 to 23, for use:
   a. as an insert to support a damaged cornea;
   b. as a transplant to replace part of the damaged cornea;
   c. as a stromal replacement, for example in the treatment of keratoconus;
   d. in re-shaping of the cornea, for example reshaping a donor cornea for transplantation;
   e. in stabilising corneal shape.
33. The use of a hydrogel according to any one of paragraphs 14 to 23, as a support for cell growth; optionally wherein
   a. the hydrogel is used as a scaffold for corneal endothelial cells (e.g. as a corneal endothelial graft material);
   b. the hydrogel is used as a support for a stratified conjunctival epithelium with embedded mucin-secreting goblet cells;
   c. the hydrogel is used as a substrate to grow retinal pigment epithelium cells.

1. A method for making a hydrogel, the method comprising:
(i) providing a solution comprising a modified poly-ε-lysine polymer which comprises a repeating monomer unit according to formula (A) below: 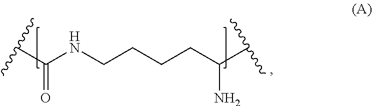
wherein from 10% to 75% of the amino groups have been functionalised with pendant functional acrylate groups; and
ii) exposing the modified poly-ε-lysine polymer to UV light to crosslink the modified poly-ε-lysine and provide the hydrogel.

2. The method according to claim 1, wherein
the functional acrylate groups have the following structure: 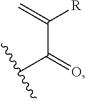
wherein R is selected from H or (1-4C)alkyl.

3. The method according to claim 1, wherein the pendent acrylate groups are selected from acrylate or methacrylate.

4. The method according to claim 1, wherein the solution is an aqueous solution; and/or
the solution comprises modified poly-ε-lysine in an amount of from 5 to 35 wt. %, optionally from 10 to 25 wt. %; and/or
the solution has a pH in the range of 6 to 8.5, optionally 7 to 8.

5. The method according to claim 1, wherein the solution further comprises a poly(ethylene glycol) diacrylate polymer.

6. The method according to claim 1, wherein the solution comprises a buffer to maintain the pH, optionally wherein the buffer is selected from phosphate buffered saline.

7.-9. (canceled)

10. The method according to claim 1, comprising casting the solution of modified poly-ε-lysine into a film prior to exposure to UV light.

11. The method according to claim 1, wherein the UV light has a wavelength of 250 to 405 nm.

12.-13. (canceled)

14. A hydrogel comprising a polymer which comprises a repeating monomer unit according to formula (A) below: 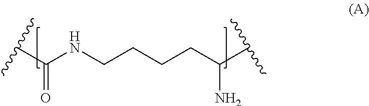
wherein from 10% to 75% of the amine groups are functionalised with an acrylate group of the formula: 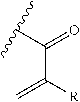
or are crosslinked by a group of the formula: 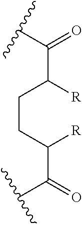
wherein each R is selected from hydrogen or (1-4C)alkyl.

15. The hydrogel according to claim 14, wherein each R is hydrogen or methyl.

16. The hydrogel according to claim 14, wherein from 30% to 75% of the amino groups have been functionalised with pendant functional acrylate groups.

17. The hydrogel according to claim 14, wherein the hydrogel is in the form of a film;
optionally wherein the hydrogel has a film thickness of from 10 to 500 microns, further optionally wherein the hydrogel has a film thickness of from 10 to 50 microns.

18. The hydrogel according to claim 14, wherein the hydrogel has a water content of at least 70 wt. %; and/or
the hydrogel has a light transmittance of at least 80%.

19. The hydrogel according to claim 14, wherein the modified poly-ε-lysine is the only gel forming component of the hydrogel.

20. The hydrogel according to claim 14, wherein the hydrogel further comprises a poly(ethylene glycol) diacrylate polymer crosslinked to the modified poly-ε-lysine of the hydrogel;
optionally wherein the weight ratio of modified poly-ε-lysine to poly(ethylene glycol) polymer in the hydrogel is from 0.5:1 to 50:1, preferably from 1:1 to 40:1, more preferably from 5:1 to 25:1.

21. The hydrogel according to claim 14, wherein the hydrogel further comprises a poly(ethylene glycol) diacrylate polymer crosslinked to the modified poly-ε-lysine of the hydrogel;
wherein the weight ratio of modified poly-ε-lysine to poly(ethylene glycol) diacrylate polymer in the hydrogel is from 40:60 to 70:30.

22. The hydrogel according to claim 20, wherein the modified poly-ε-lysine and poly(ethylene glycol) polymer are the only gel forming components of the hydrogel.

23. A bandage contact lens comprising a hydrogel according to claim 14.

24.-28. (canceled)

29. A method of treating disease or damage to the corneal epithelium or corneal endothelium, said method comprising applying a hydrogel according to claim 14 to the eye of a subject in need of such treatment; optionally wherein:
a. the disease or damage to the corneal epithelium or corneal endothelium comprises any corneal epithelial defect (for example persistent corneal defects, microbial keratitis, damage following corneal surgery or in post corneal cross-linking for keratoconus) or ocular epithelial dysfunction (for example limbal stem cell deficiency, microbial keratitis);
b. the disease or damage to the corneal epithelium or corneal endothelium comprises any corneal endothelial dysfunction, for example in the treatment of corneal swelling due corneal oedema (e.g. bullous keratopathy, Fuchs Endothelial Dystrophy, Congenital Hereditary Endothelial Dystrophy, hydrops of the cornea in keratoconus).
c. the hydrogel is used as an insert to support a damaged cornea;
d. the hydrogel is used as a transplant to replace part of the damaged cornea;
e. the hydrogel is used as a stromal replacement, for example in the treatment of keratoconus;
f. the hydrogel is used in re-shaping of the cornea, for example reshaping a donor cornea for transplantation; or
g. the hydrogel is used in stabilizing corneal shape.

30. (canceled)

31. A biomaterial substrate suitable to act as a support for cell growth, wherein the biomaterial substrate comprises the hydrogel according to claim 14; optionally wherein:
a. the hydrogel is used as a scaffold for corneal endothelial cells (e.g. as a corneal endothelial graft material);
b. the hydrogel is used as a support for a stratified conjunctival epithelium with embedded mucin-secreting goblet cells; or
c. the hydrogel is used as a substrate to grow retinal pigment epithelium cells.
